(19) United States
(12) Patent Application Publication
(10) Pub. No.: US 2024/0024678 A1
(43) Pub. Date: Jan. 25, 2024

(54) FABRICATION AND APPLICATION OF ELECTROCEUTICAL SYSTEMS USING SMART PHOTONIC LENS

(71) Applicants: PHI BIOMED INC., Seoul (KR); POSTECH RESEARCH AND BUSINESS DEVELOPMENT FOUNDATION, Gyeongsangbuk-do (KR)

(72) Inventors: Sei Kwang HAHN, Seoul (KR); Sang Bale SHIN, Gyeongsangbuk-do (KR)

(21) Appl. No.: 18/201,142

(22) Filed: May 23, 2023

Related U.S. Application Data
(63) Continuation-in-part of application No. 16/642,979, filed on Feb. 28, 2020, now abandoned.

(51) Int. Cl. A61N 1/36; A61N 1/378; A61N 1/05; A61N 1/372

(52) U.S. Cl. CPC A61N 1/36046; A61N 1/3787; A61N 1/0543; A61N 1/36025; A61N 1/37235

(57) ABSTRACT

The present invention relates to an electroceutical system for driving a photoelectric element implanted in a sub-retinal optic nerve using a smart photonic lens. According to the present invention, by artificially emitting the light of the smart photonic lens to a photoelectric element connected to the optic nerve, it is possible to utilize stimulation of the nerve with an electric current generated in the photoelectric element to treat various diseases.

### RELATED APPLICATION

This application is a continuation-in-part of and claims the benefit of co-pending U.S. patent application Ser. No. 16/642,979 filed Feb. 28, 2020.

### STATEMENT REGARDING SPONSORED RESEARCH OR DEVELOPMENT

This research was supported by the Center for Advanced Soft-Electronics (Global Frontier Project, CASE-2015M3A6A5072945, 30%) of the National Research Foundation (NRF) funded by the Ministry of Science and ICT, Korea. This work was also supported by the World Class 300 Project (S2482887, 70%) funded by the Ministry of SMEs and Startups, Korea.

### TECHNICAL FIELD

The present invention relates to an electroceutical system using a smart photonic lens.

### BACKGROUND ART

Recently, research on electroceuticals for treating diseases by stimulating nerves through electrical stimulation has been actively conducted worldwide (Patent Document 1).

Various studies are being conducted to apply such electroceuticals to almost all diseases, including brain diseases such as Alzheimer's and Parkinson's diseases: metabolic diseases such as diabetes, obesity, and hypertension; arthritis; hepatitis; inflammatory diseases; and optic nerve diseases. However, most of the electroceuticals are based on an invasive procedure involving an implantation treatment. For the electroceuticals, a surgery of transplantation into a patient's body is required, it is difficult to supply power for driving in the body, and an available period is limited.

### PATENT DOCUMENT

U.S. Patent Publication No. 2018-0071535

### DISCLOSURE

### Technical Problem

According to the present invention, in order to solve the above-described problems of electroceuticals, there is provided an electroceutical system for driving a photoelectric element of an electroceutical device implanted in a sub-retinal optic nerve using a contact lens including a light-emitting diode (LED) light source.

By artificially emitting the light of the contact lens to a sub-retinal photoelectric element connected to the optic nerve, the present invention is directed to utilizing stimulation of the nerve with an electric current generated in the photoelectric element to treat various diseases.

### Technical Solution

The present invention provides an electroceutical system including a contact lens including a light-emitting diode (LED) light source; and an electroceutical device.

The electroceutical device is implanted in a sub-retinal optic nerve.

The electroceutical device is configured to convert light emitted from the LED light source into an electric signal.

The present invention provides a method of driving an electroceutical system, the method including causing: a light-emitting diode (LED) light source in a contact lens to emit light to an electroceutical device within a predetermined time period; and
causing a photoelectric element of the electroceutical device to convert the emitted light into an electric signal, generate an electric current, and stimulate an optic nerve,
wherein the electroceutical device is implanted in a sub-retinal optic nerve.

The present invention also provides a method of treating a disease using the above-described electroceutical system.

### Advantageous Effects

According to the present invention, by artificially emitting light such as visible light or infrared light generated in a light-emitting diode (LED) light source of a contact lens to an electroceutical device connected to an optic nerve, it is possible to utilize stimulation of the nerve with an electric current generated in a photoelectric element of the electroceutical device to treat various diseases.

The present invention has an advantage in that the electroceutical device can be driven even without a separate power supply source. Also, since light is emitted through the LED light source included in the contact lens, the light can stably reach the electroceutical device by adjusting the location of the LED light source in the lens, and the electroceutical system can be easily used without being influenced by time and place. Also, since the LED light source is used, the present invention has an advantage in that a light source is easily selected for each wavelength and in that the amount of light is adjustable.

The electroceutical system of the present invention is applicable to various diseases that can be treated through nerve stimulation, the diseases including brain diseases such as Alzheimer's and Parkinson's diseases; metabolic diseases such as diabetes, obesity, and hypertension; arthritis; infections; inflammatory diseases; and optic nerve diseases.

### DESCRIPTION OF DRAWINGS

FIG. 1 shows an electroceutical system using a light-emitting diode (LED) light source 10 of a contact lens 11 according to the present invention.

FIG. 2 shows a design and production example of an application-specific integrated circuit (ASIC) 20 according to the present invention.

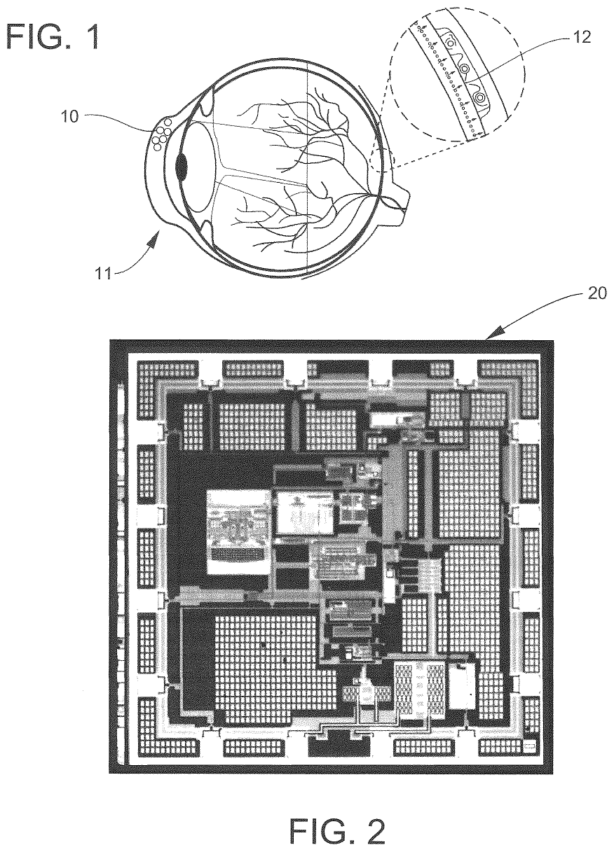

In an embodiment, the LED light source may emit light to a retina. A subretinal electroceutical device 12 (see expanded view of FIG. 1) may convert the emitted light into an electronic signal, and thus the electronic signal may be applied to disease treatment.

The method of fabricating the contact lens is shown in FIG. 3, As shown in FIG. 3, the contact lens of the present invention was fabricated through a process of Metal Deposition 31, Photolithography 32, Flip-Chip Bonding 33, LED Bonding 34, and Contact Lens 35.

In the present invention, FIG, 4 illustrates a blueprint of a contact lens according to the present invention. As shown in FIG. 4, according to the present invention, a contact lens 11 including an LED light source 10, an application-specific integrated circuit (ASIC chip) 41, an antenna 42, etc. may be fabricated.

FIG. 5 shows a gold pad 51 produced on a flexible transparent substrate according to the present invention.

FIG. 6A shows a flexible transparent substrate on which application-specific integrated circuit and the like are patterned on and bonded; FIG. 6B shows an application-specific integrated circuit flip-chip bonded to the gold pad of FIG. 5; FIG. 6C shows a transparent substrate with Ag paste printed on it; FIG. 6D shows the printed Ag paste of FIG. 6C in detail; FIG. 6E shows a transparent substrate on which electronic elements such as an LED light source, condenser, battery, and resistor are bonded; FIG. 6F shows an image that confirms the operational status of the bonded electronic elements.

FIG. 7A shows an image of a commercial photodiode; FIGS. 7B and 7C show an image of multiple gold bumps; FIG. 7D shows a subminiature photovoltaic element fabricated on a flexible substrate. Coins were used to show the size of the photovoltaic element.

FIG. 8A shows a design of a subminiature module produced on a PCB substrate; FIG. 8B shows an image of the manufactured subminiature module; FIG. 8C shows an antenna that can be connected to a subminiature module through a connector. Coins were used to compare the size of the antenna.

FIG. 9A shows an image of a contact lens including an LED light source, an application-specific integrated circuit, and an antenna; FIG. 9B shows a photo confirming that the contact lens can be driven by directly connecting the module to the antenna of the contact lens; FIG. 9C shows a photo of a cable connected to the antenna of the contact lens, and you can check the operation of the contact lens via the cable.

FIG. 10 illustrates an example of applying a contact lens to an animal according to the present invention.

FIG. 11 shows a photocurrent measuring result of an electroceutical system using a contact lens and a photoelectric element according to the present invention.

FIG. 12 illustrates the concept of the electroceutical system. In the intended LED light source, light energy is converted into electrical energy through an implanted device (electroceutical device), and electrical stimulation is performed by electrodes connected to the optic nerve.

FIG. 13 illustrates a process of electroceutical system. LED light reaching the light receiver converted into electrical energy through the photoelectric element, and electrical stimulation is performed on electrodes connected to the optic nerve.

FIG. 14 illustrates a structure of implant device (electroceutical device). The light receiver more efficiently transfers the arriving light to the photoelectric element, and the photoelectric element performs efficient light-to-electrical energy conversion. For connection with the optic nerve, the electrode of the implant device is configured in the form of a microneedle.

### BEST MODE FOR CARRYING OUT THE INVENTION

The present invention relates to an electroceutical system including a contact lens including a light-emitting diode (LED) light source; and an electroceutical device.

The electroceutical device is implanted in a sub-retinal optic nerve.

The electroceutical device is configured to convert light emitted from the LED light source into an electric signal.

Hereinafter, the electroceutical system of the present invention will be described in detail.

The electroceutical system according to the present invention may be used to treat a disease that can be treated through nerve stimulation.

The disease that can be treated through nerve stimulation is not particularly limited and may be selected from the group consisting of, for example, brain diseases such as Alzheimer's and Parkinson's diseases; metabolic diseases such as diabetes, obesity, and hypertension; arthritis; infections; inflammatory diseases; and optic nerve diseases. In this case, the treatment of optic nerve diseases refers to a vision treatment.

The electroceutical system of the present invention includes a contact lens and an electroceutical device.

In the present invention, the contact lens may be based on one or more polymers selected from the group consisting of silicone elastomers; silicone hydrogels; polydimethylsiloxane (PDMS); poly(2-hydroxyethyl methacrylate) (PHEMA); and polyethylene glycol) methacrylate (PEGMA).

In the present invention, the contact lens (hereinafter referred to as a smart lens) includes an LED light source.

Light in everyday life includes visible and ultraviolet light, and the amount of light penetrated into a human body or eye is very small. Red light rays and infrared light rays can penetrate up to several centimeters and thus may be used to treat cells in a human body.

According to the present invention, such light rays are applied to a contact lens and stably delivered up to an optic nerve. For example, when the LED light source included in the contact lens is located at the center of a pupil to emit light, light rays such as ultraviolet light rays, blue light rays, green light rays, and/or red light rays may stably reach an optic nerve.

In an embodiment, the LED light source may be a micro-LED (mLED or μLED).

A product commonly available in the art or a product produced by hand may be used as the LED light source, that is, the micro-LED.

In an embodiment, the LED light source may emit light to a retina. A sub-retinal electroceutical device may convert the emitted light into an electronic signal, and thus the electronic signal may be applied to disease treatment.

In an embodiment, the LED light source may selectively include LEDs that emit light of specific wavelengths according to a use purpose. For example, the LED light source may include one or more light sources selected from the group consisting of an ultraviolet light source, a blue light source, a green light source, a red light source, and an infrared light source.

Also, in an embodiment, the position of the LED light source in the contact lens is not particularly limited and may be appropriately adjusted. For example, the position of the LED light source may be adjusted according to the position of an electroceutical device implanted under the retina. In detail, the LED light source may be positioned near the center of the pupil.

In the present invention, the LED light source can be artificially driven and the position of the LED light source can be adjusted. Thus, by selecting a light source for each wavelength and adjusting the amount of light, it is possible to make light (light rays) reach up to a desired position in an eyeball.

In an embodiment, a transparent substrate may be formed inside the contact lens, and the LED light source may be formed on the transparent substrate.

The transparent substrate may be excellent in light transmittance, flexible, and extensible. Also, the transparent substrate may have excellent biocompatibility. The transparent substrate may contain one or more selected from the group consisting of Parylene C, PDMS, silicone elastomers, polyethylene terephthalate (PET), and polyimide (PI).

In an embodiment, the LED light source may be formed on a surface of the transparent substrate toward the eyeball.

In addition to the above-described LED light source, the contact lens of the present invention may additionally include one or more selected from the group consisting of an application-specific integrated circuit (ASIC), a battery, and an antenna.

In an embodiment, the ASIC may be used to perform wireless control, power transmission, and the like of the LED light source. The ASIC may include (1) a digital controller, (2) a relaxation oscillator, (3) a carrier frequency generator, (4) a bandgap reference generator, (5) a Vdd generator, and the like. The ASIC may be produced and used according to an intended usage.

In an embodiment, the battery may be a thin-film battery that is re-chargeable and flexible. By using the thin-film battery, it is possible to wirelessly drive the contact lens, and it is also possible to implement a system that can operate without external power.

The battery may supply power to the elements of the contact lens. Also, the battery may be prevented from being broken despite repeated bending or deformation, may be sealed when the battery is applied to the lens, and also may ensure stability in an eyeball. A product commonly available in the art or a product produced by hand may be used as the thin-film battery.

In an embodiment, the antenna may transmit and receive power and signals from and to the outside through induced current and electromagnetic resonance. The antenna may be a circular antenna having a circular structure.

The antenna may contain a nanomaterial, and the nanomaterial may include one or more selected from the group consisting of a zero-dimensional material such as nanoparticles, a one-dimensional nanomaterial such as nanowires, nanofibers, or nanotubes, and a two-dimensional nanomaterial such as graphene, MoS₂, or nanoflakes.

In an embodiment, the antenna may include a wireless electric antenna for receiving power generated by an external source, that is, wireless power and a radio frequency antenna for performing data communication.

In particular, according to the present invention, a wireless electric antenna may be used to complement the role of the battery. The wireless electric antenna may receive power generated by a wireless electric coil of smart glasses, which will be described below. The received power may be used to drive the LED light source through the control of the ASIC.

In an embodiment, the ASIC, battery, and antenna which have been described above may be formed on the transparent substrate to facilitate production and driving. The ASIC, battery, and antenna may be formed on the surface of the transparent substrate toward the eyeball, that is, on the same surface as that of the LED light source.

The electroceutical system of the present invention includes an electroceutical device.

In the present invention, the electroceutical device refers to a device that is implanted in a patient to provide electrical stimulation to the patient's nerves to treat the patient's disease and/or disorder.

In an embodiment, the electroceutical device is implanted in a sub-retinal optic nerve and may be connected to the optic nerve (optic nerve tissue).

In an embodiment, the electroceutical device (implant device) comprises, a light receiver, a photoelectric element, an electrodes for optic nerve stimulation.

The light receiver more efficiently transfers the arriving light from the LED light source to the photoelectric element. The photoelectric element functions to convert light (light rays) into an electronic signal and may generate current even when there is no separate voltage or current source.

The electrodes may be connected to the optic nerve tissue, In detail, a bump positioned on a line connected to a negative (−) electrode and/or a positive (+) electrode of the electroceutical device may be connected to the optic nerve. In this case, a gold bump may be used as the bump. The connection may be performed through a usual method in the art.

By operatively associating the photoelectric element connected to the optic nerve tissue, that is, the electroceutical device with the contact lens, it is possible to artificially impart electrical stimulation to an optic nerve. Accordingly, the electroceutical device of the present invention may be expressed as a photoelectric implant.

In an embodiment, the electroceutical device does not need a separate power source and a separate circuit for driving an invasive element and may include only a photoelectric element, which is a single element, and a connection part to control necessary electric current stimulation.

In order to reach the implanted device from a light source of the intended light intensity, wavelength and frequency, the device is implanted at the fovea centralis site. In order to efficiently collect light energy, an optical lens is formed on the optoelectronic part, and a biocompatible epoxy is generally used. The converted electrical signal is connected to the optic nerve through a microneedle type electrode. The electrode of the implant device is composed of Polycaprolactone (PCL), which is a biocompatible polymer material, and vaccum deposited gold surface.

Also, the electroceutical device of the present invention may additionally include smart glasses.

According to the present invention, the smart glasses may wirelessly transmit or receive electrical signals to adjust the driving of the LED light source of the contact lens. The smart glasses may use a rechargeable lithium ion battery as a power source and may perform wireless communication with a smart device using a Bluetooth module installed therein.

The smart glasses may be paired with a smartphone, a smartwatch, or a personal computer (PC). The smart glasses may use a lithium ion cell embedded therein as power, and a photocell may be inserted into the smart glasses for the purpose of self-powering. The smart glasses may have a total weight of less than 20 g and may support Wi-Fi 802.11b/g, Bluetooth, and micro USB.

Also, the present invention relates to a method of fabricating the above-described electroceutical system. As described above, the electroceutical system includes a contact lens and an electroceutical device.

In the present invention, when an LED light source is configured on a stretchable substrate, the fabrication method may include: forming a sacrificial layer to be dissolved in water on a handling substrate (S1);
forming a transparent substrate on the sacrificial layer (S2);
forming an LED light source on the transparent substrate (S3); and
transferring the transparent substrate on which the LED light source is formed into a contact lens (S4).

Operation S1 is an operation of forming a sacrificial layer on a handling substrate.

The sacrificial layer may serve as an adhesive layer between the handling substrate and the transparent substrate and may help transfer the transparent substrate on which the LED light source is formed. Such a sacrificial layer is not particularly limited as long as it can be dissolved in water, and may include one or more selected from the group consisting of polyvinyl alcohol (PVA) and dextran.

Operation S2 is an operation of forming a transparent substrate on the sacrificial layer. Here, the sacrificial layer serves as an adhesive. Accordingly, the transparent substrate may be easily attached to the handling substrate and may be easily detached from the handling substrate through dissolution of the sacrificial layer in a following process.

In an embodiment, for the transparent substrate, a material having excellent light transmittance may be used, and the above-described types of materials may be used.

Operation S3 is an operation of forming an LED light source on the transparent substrate.

In an embodiment, the LED light source may be bonded to the transparent substrate using a biocompatible epoxy, e.g., Ag epoxy, etc.

Also, operation S4 is an operation of transferring the transparent substrate on which the LED light source is formed into a contact lens.

The LED light source produced on the sacrificial layer may be transferred while melting the sacrificial layer in biocompatible water.

Also, the present invention may additionally include an operation of forming an application-specific integrated circuit, a battery, and an antenna on the transparent substrate. The operation may be performed while operation S3 is performed.

In an embodiment, the application-specific integrated circuit may be fabricated through an operation of depositing a metal such as gold or aluminum on the transparent substrate and then forming a metal pad through an etching method using a photolithography process; and
an operation of bonding a device to the metal pad through a flip-chip bonding process.

In the flip-chip bonding process, a non-conductive adhesive may be used to bond the device through an ultrasonic and thermal compression process.

In an embodiment, the battery may be formed on the transparent substrate in the same way as that of the LED light source.

Also, in an embodiment, the antenna may be fabricated through an operation of forming a mask material for patterning on the transparent substrate (a1);
an operation of patterning a sensor and a circuit by coating the transparent substrate on which the mask material is formed with a nanomaterial through a lift-off process (a2); and
an operation of forming a passivation layer on the patterned sensor and circuit (a3).

Operation a1 is an operation of forming a mask material for patterning on the transparent substrate.

The mask material may serve as a shadow mask, and the nanomaterial may be patterned by using the mask material. A material that can be used as a photoresist may be used as the mask material. In detail, LOF, AZ series, and the like may be used.

Operation a2 is an operation of patterning a sensor and a circuit by coating the transparent substrate on which the mask material is formed with the nanornaterial through a lift-off process.

Through the above operation, a nanomaterial pattern may be formed. As the nanomaterial, the above-described types of materials may be used. In detail, a silver nanowire may be used.

The nanornaterial fabricated in the above operation may act as an antenna.

Also, the circuit fabricated in the above operation may serve to connect the LED light source, the semiconductor device, the antenna, and the battery.

Operation a3 is an operation of forming a passivation layer on the patterned antenna and circuit.

By forming the passivation layer, it is possible to prevent the loss of nanomaterials and improve electrical stability.

In the present invention, the electroceutical device may be fabricated by packaging a photoelectric element for the purpose of insertion into human bodies. In this case, a biocompatible resin may be used as the packaging material, and ethylene vinyl acetate (EVA), polyurethane (PUR), polyacrylonitrile (PAN), or polyvinyl chloride (PVC) may be used as the biocompatible resin. Upon the packaging, light waveguide processing may be performed in consideration of an antireflection coating processing part deposited on a light absorption part (window) in order to prevent deterioration of photocurrent efficiency.

Also, the present invention relates to a method of driving the above-described electroceutical system.

The driving method may include: causing the LED light source in the contact lens to emit light to the electroceutical device within a predetermined time period; and
causing the photoelectric element of the electroceutical device to convert the emitted light into an electric signal to generate an electric current to stimulate an optic nerve.

In an embodiment, the LED light source of the contact lens may emit light to an electroceutical device implanted in a sub-retinal optic nerve within a predetermined time period. Also, the photoelectric element of the electroceutical device may convert the emitted light into an electric signal to generate an electric current and stimulate an optic nerve (see FIG. 1). In this case, the driving or control of the LED light source may be performed by an application-specific integrated circuit.

In an embodiment, the electroceutical system may additionally include smart glasses. Wireless power which is power generated in a wireless electric coil of the smart glasses may be received by a wireless electric antenna of the contact lens, and power received through control of the application-specific integrated circuit may be used to drive the LED light source.

Also, the present invention relates to a method of treating a disease using the above-described electroceutical system.

In the present invention, the photoelectric element of the electroceutical device may convert light emitted from the LED light source of the contact lens into an electric signal, generate an electric current, and stimulate an optic nerve to treat a disease.

The disease is a disease that can be treated through nerve stimulation and may be selected from the group consisting of, for example, brain diseases such as Alzheimer's and Parkinson's diseases; metabolic diseases such as diabetes, obesity, and hypertension; arthritis; infections; inflammatory diseases; and optic nerve diseases. In this case, the treatment of optic nerve diseases means a vision treatment.

### MODE FOR CARRYING OUT THE INVENTION

The present invention will be described in detail below with reference to the following embodiments. However, the following embodiments are merely illustrative of the present invention, and the present invention is not limited to the following embodiments.

### Embodiments

### Fabrication Example 1: Fabrication of Contact Lens

(1) Design and Production of Application-Specific Integrated Circuit

For the purpose of wireless control and electric power transmission of the LED light source, there is a need for an application-specific integrated circuit including a circuit having (1) a digital controller, (2) a relaxation oscillator, (3) a carrier frequency generator, (4) a bandgap reference generator, (5) a Vdd generator, and the like. It is possible to deliver and drive wireless power of the contact lens using the application-specific integrated circuit, and also it is possible to control electric current and light emission timing. The light source may apply ultraviolet, blue, green, red, and infrared light-emitting LEDs.

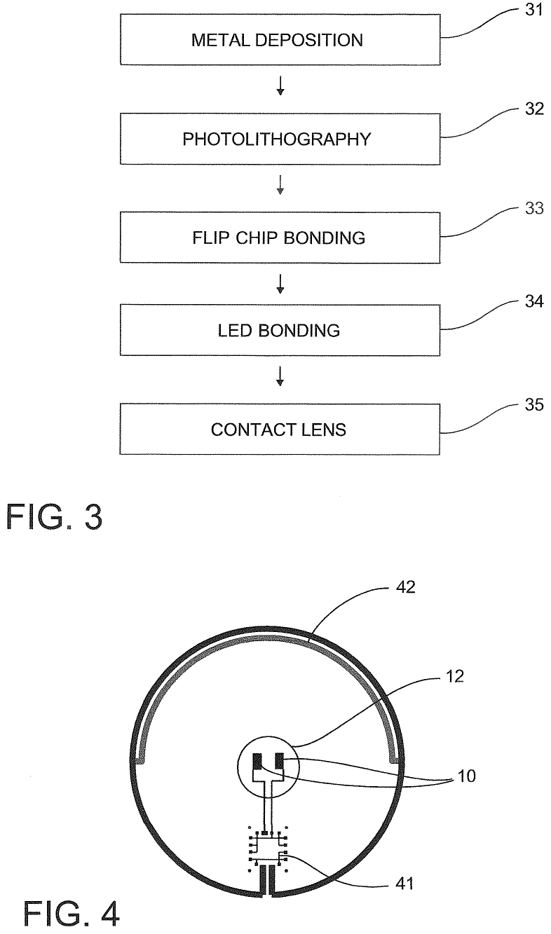

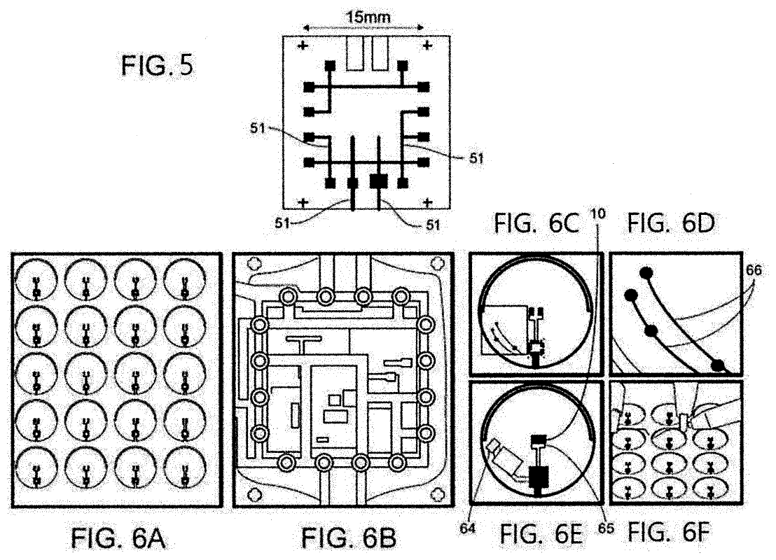

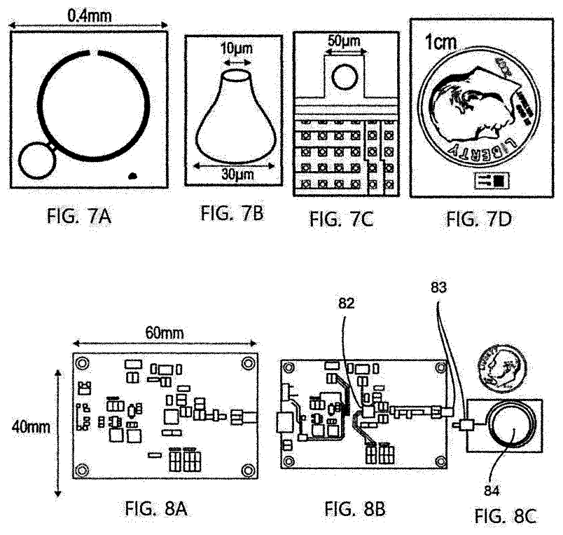

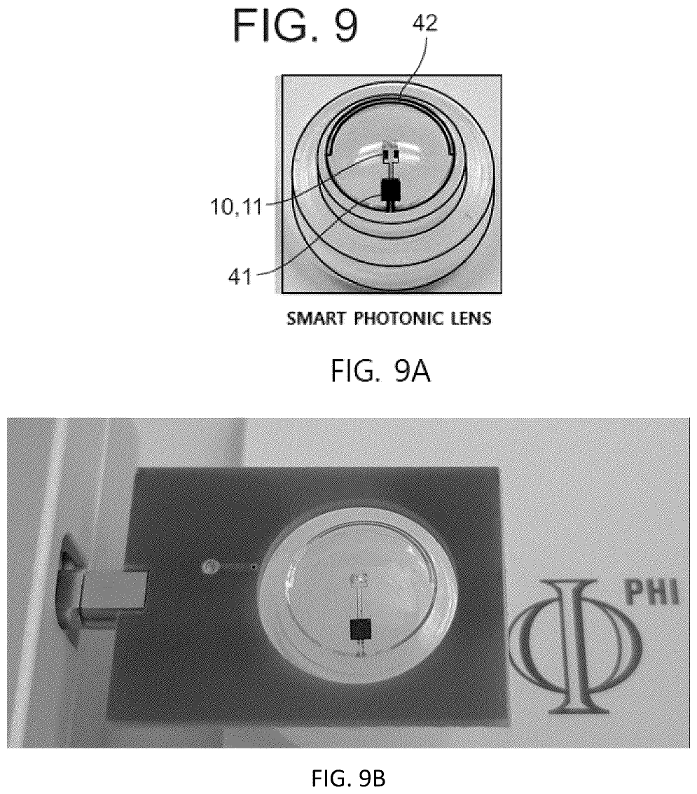

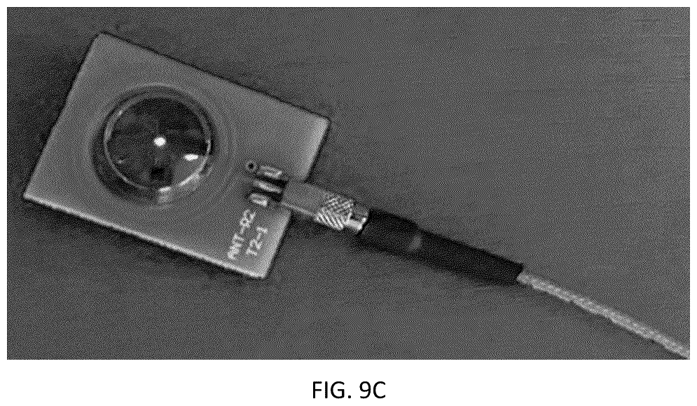

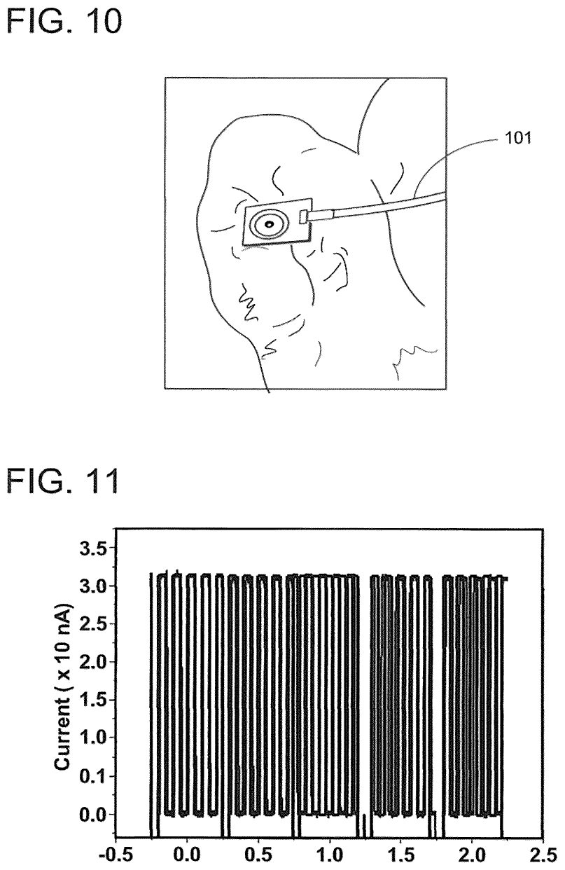

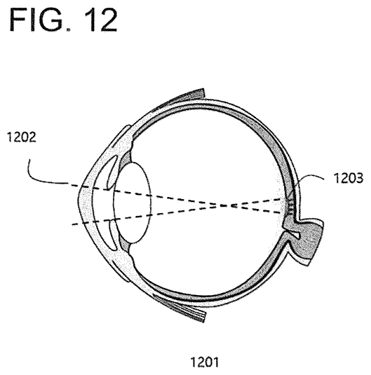

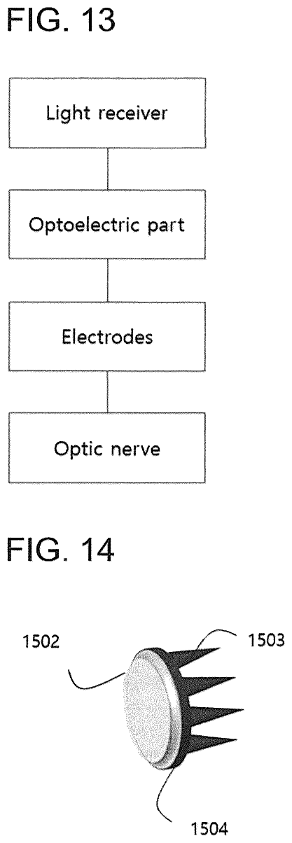

First, the application-specific integrated circuit may be produced through an operation for computer simulation, layout generation, and TOAD simulation and was produced through a CMOS process (0.18 μm or less) in consideration of its own power consumption (See FIG. 2).

(2) Fabrication of Contact Lens

The contact lens was produced using the LED light source and the application-specific integrated circuit fabricated in (1).

The method of fabricating the contact lens is shown in FIG. 3. As shown in FIG. 3, the contact lens of the present invention was fabricated through a process of Metal Deposition, Photolithography, Flip-Chip Bonding, LED Bonding, and Contact Lens.

In detail, a metal such as gold or aluminum was deposited to a thickness of 200 to 500 nm on a flexible transparent substrate having 30 μm or less, and then a pad was formed using a wet/dry etching method that uses a photolithography process. Then, by using a flip-chip bonding process, the application-specific integrated circuit was bonded to the flexible transparent substrate using a non-conductive adhesive through an ultrasonic and thermal compression process. The LED light source, the battery, a condenser and resistor for controlling voltage and current, and the like were bonded using a biocompatible epoxy, e.g., Ag epoxy in consideration of the heat resistance of the flexible plastic substrate.

Only elements were cut out of the transparent substrate to which the elements are bonded by using a laser cutter or the like, and then a lens was produced using biocompatible silicon (Si) elastomer or the like.

Subsequently, a contact lens was driven through an antenna and a driving board having an RF transmission processing function.

In the present invention, FIG. 4 illustrates a blueprint of a contact lens according to the present invention. As shown in FIG. 4, according to the present invention, a contact lens including an LED light source, an application-specific integrated circuit (ASIC chip), an antenna, etc. may be fabricated.

Also, FIG. 5 shows a gold pad produced on a flexible transparent substrate according to the present invention. A semiconductor element may be easily bonded to the gold pad through a flip-chip bonding process.

Also, FIG. 6 shows photos after flip-chip bonding performed on a flexible transparent substrate (left and central photos) and a photo after Ag epoxy bonding of an LED light source or the like (a right photo). Referring to FIG. 6, the flip-chip bonding result of the application-specific integrated circuit patterned on and bonded to the transparent substrate may be checked. Also, electronic elements such as an LED light source, condenser, battery, and resistor may be bonded using Ag epoxy, and then an operational status may be checked.

### Fabrication Example 2: Production of Sub-retinal Electroceutical Device

As a photoelectric element, a commercially available high-performance photodiode was used, and an element having a structure optimized according to the wavelength of a light source was used. Also, as a photodiode, a product ranging in size from several tens of micrometers to several millimeters was used depending on the purpose and necessary electric current.

A packaging process using a biocompatible resin was performed to insert the photoelectric element into a human body. Upon the packaging process, light waveguide processing was performed in consideration of an antireflection coating processing part deposited on a light absorption part (window) in order to prevent deterioration of photocurrent efficiency. Also, a fine gold bump was formed to connect to optic nerve tissue and was multi-connected to the photoelectric device.

In the present invention, FIG. 7 shows examples of a commercially available photodiode (a left photo), formation of multiple gold bumps (two central photos), and production of a subminiature photoelectric element on a flexible substrate (a right photo).

As shown in FIG. 7, a photodiode formed on a flexible transparent substrate and a gold bump configured to connect to an optic nerve can be checked.

### Fabrication Example 3: Development of Flexible Subminiature Module

A subminiature module was designed with a circuit configuration for essential components such as a photoelectric element, a signal amplifier, a wireless module, and a battery, and data processing, calibration, and mode control functions were processed by software.

When a device was provided as a printed circuit board (PCB) or a flexible printed circuit board (FPCB; polyimide), the device was produced with a size of at least 20 cm² and was produced as a band-type module. In the case of an eyeglass module, the aspect ratio of the module may be flexibly adjusted depending on an application part.

FIG. 8 shows an example of the design and fabrication of a subminiature module produced on a PCB substrate.

As shown in FIG. 8, the subminiature module may be produced as a PCB module and may have an antenna adjustable in location by using a wireless coaxial cable depending on a purpose. The module may be operated by internal battery or USB power.

Also, FIG. 9 illustrates an example of driving a contact lens.

The left photo in FIG. 9 is a photo of a contact lens including an LED light source, an application-specific integrated circuit, and an antenna. By connecting the module to the antenna of the contact lens fabricated according to this embodiment directly or by connecting the module to a cable through the center and right photos, it can be seen that the contact lens can be driven.

Also, FIG. 10 illustrates an example of applying a contact lens to an animal according to the present invention.

A contact lens experiment was conducted on a lab rabbit, and the operation of a contact lens including a red LED light source that can be wirelessly driven through the PCB module and cable can be verified.

### Experimental Example 1: Measurement of Photocurrent Generated in Photoelectric Element by LED Light Source of Contact Lens

(1) Method

Using a wireless driving module, the photoelectric element and the contact lens including the red LED light source were driven.

In detail, in the present experimental example, a permeability test for sheep blood contained in a quartz cuvette was performed using the red LED light source of the contact lens. The experiment was performed after a blood sample was spaced 2 cm apart from the light source and the photoelectric element.

(2) Result

The measurement result was shown in FIG. 11.

FIG. 11 shows a result of measuring a photoelectric current generated in the photoelectric element by the red LED light source of the contact lens.

As shown in FIG. 11, the photoelectric current generated through blood present in the cuvette was found to be about 30 nA. It can be seen that the photoelectric current is proportional to the distance from the light source and the size of the photodiode.

### INDUSTRIAL APPLICABILITY

The electroceutical system of the present invention is applicable to various diseases that can be treated through nerve stimulation, the diseases including brain diseases such as Alzheimer's and Parkinson's diseases; metabolic diseases such as diabetes, obesity, and hypertension; arthritis; infections; inflammatory diseases; and optic nerve diseases.

### DESCRIPTION OF THE SYMBOLS

10: light-emitting diode (LED) light source, 11: contact lens, 12: subretinal electroceutical device, 20: application-specific integrated circuit (ASIC), 31: process of Metal Deposition, 32: Photolithography, 33: Flip-Chip Bonding, 34: LED Bonding, Contact Lens, 41: application-specific integrated circuit (ASIC chip), 42: antenna, 51: gold pad, 64: BAT, 65: RES, 66: Printed Ag paste, 82: subminiature module, 83: connector, 84: antenna, 1201: concept of electroceutical system, 1202: intended LED light, 1203: implant device, 1502: light receiver, 1503: electrodes for optic nerve stimulation, 1504: photoelectric element

1. An electroceutical system comprising:
a contact lens comprising a light-emitting diode (LED) light source; and
an electroceutical device,
wherein, the electroceutical device comprises, a light receiver, a photoelectric element, an electrodes for optic nerve stimulation,
the photoelectric element converts light emitted from the LED light source into an electric signal, the electrodes is configured to be connected to sub-retinal optic nerve tissue to stimulate the nerve with an electric current generated in the photoelectric element, and
the electroceutical system is used to treat a disease curable through nerve stimulation.

2. The electroceutical system of claim 1, wherein the disease is selected from the group consisting of brain diseases such as Alzheimer's and Parkinson's diseases; metabolic diseases such as diabetes, obesity, and hypertension; arthritis; infections; inflammatory diseases; and optic nerve diseases.

3. The electroceutical system of claim 1, wherein the contact lens is made from one or more selected from the group consisting of silicone elastomers; silicone hydrogels; polydimethylsiloxane (PDMS); poly(2-hydroxyethyl methacrylate) (PHEMA); and polyethylene glycol) methacrylate (PEGMA).

4. The electroceutical system of 1, wherein,
the LED light source is formed on a transparent substrate, and
the transparent substrate contains one or more selected from the group consisting of Parylene C, PDMS, silicone elastomers, polyethylene terephthalate (PET), and polyimide (PI).

5. The electroceutical system of 1, wherein the contact lens further comprises one or more selected from the group consisting of an application-specific integrated circuit, an antenna, and a battery.

6. The electroceutical system of 1, wherein a bump located on a line extending from an electrode of the photoelectric element is connected to the optic nerve tissue.

7. The electroceutical system of claim 1, further comprising smart glasses, wherein the electroceutical system is driven through an electrical signal transmitted from the smart glasses.

8. A method of driving an electroceutical system to treat a disease curable through nerve stimulation, the method comprising:
causing a light-emitting diode (LED) light source in a contact lens to emit light to an electroceutical device within a predetermined time period; and
causing a photoelectric element of the electroceutical device to convert the emitted light into an electric signal, generate an electric current, such that the electric current could stimulate an optic nerve,
the electroceutical device comprises, a light receiver, a photoelectric element, an electrodes for optic nerve stimulation,
the photoelectric element converts light emitted from the LED light source into an electric signal, the electrodes is configured to be connected to sub-retinal optic nerve tissue to stimulate the nerve with an electric current generated in the photoelectric element.

9. The method of claim 8, wherein the driving of the LED light source in the contact lens is controlled by an application-specific integrated circuit.

10. The method of claim 8, wherein,
the electroceutical system further comprises smart glasses,
wireless power generated in a wireless electric coil of the smart glasses is received by an antenna of the contact lens, and
power received through control of an application-specific integrated circuit is used for driving the LED light source.
